(19) United States
(12) Patent Application Publication
(10) Pub. No.: US 2010/0273217 A1
(43) Pub. Date: Oct. 28, 2010

(54) BIOCABURANT PREPARATION USING PENCILLIUM FUNICULOSUM ENZYMES

(76) Inventor: Marc Maestracci, Neris les Bains (FR)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/745,937

(22) PCT Filed: Dec. 4, 2008
(86) PCT No.: PCT/IB2008/003714

§ 371 Date: Jun. 30, 2010

Related U.S. Application Data
(60) Provisional application No. 60/996,799, filed on Dec. 5, 2007.

(51) Int. Cl. C12P 19/00; C12P 7/10; C12N 9/24

(52) U.S. Cl. 435/72; 435/165; 435/200

(57) ABSTRACT

The present invention deals with a method for treating biomass comprising the steps of providing an enzyme mixture obtained from Penicillium funiculosum deposited under Budapest treaty in the International Mycological Institute under the number IMI 378536, providing plant biomass then contacting the enzyme mixture of step (a) and the biomass of step (b) under conditions wherein the saccharification of the biomass occurs.

The present invention deals with enzymatic saccharification of biomass for bioethanol production by an enzyme mixture obtained from Penicillium funiculosum deposited under Budapest Treaty in the International Mycological Institute under the number IMI 378536.

In particular the invention is directed to a method for treating biomass for bioethanol production with cellulase, β-glucanase, cellobiohydrolase, β-glucosidase and optionally xylanase.

Bioconversion of renewable lignocellulosic biomass to ethanol as an alternative to liquid fuels has been extensively studied in the last decades.

Bioethanol production costs are high and the energy output is low, and there is continuous research for making the process more economical. Enzymatic hydrolysis is considered the most promising technology for converting cellulosic biomass into fermentable sugars. The cost of the enzymatic step is one of the major economical factors of the process. Efforts have been made to improve the efficiency of the enzymatic hydrolysis of the cellulosic material.

Vidmantiene et al. (2006) describe a method for hydrolysing the polysaccharides from cereal derived waste to yield sugar feedstock suitable for fermentation into ethanol using two subsequent enzyme preparations the first one for starch hydrolysis and saccharification comprising α-amylase from Bacillus subtillis and β-glucanase. This step is carried out at 65° C. during 90 minutes. The second enzyme preparation comprises glucoamylase from Aspergillus awamori, α-amylase and β-glucanase and β-xylanase, cellulase and β-glucanase from Trichoderma reesei and is used at a temperature of 55-60° C. during 120 minutes.

Ohgren et al. (2006) showed that a prehydrolysis treatment has no or negative effect on the overall ethanol yield, said pretreatment being made during 16, 8 or 4 hours either with commercial cellulase mixture supplemented with β-glucosidase at 48° C. or a developmental mixture of thermoactive enzymes at 55° C. consisting of a modified cellobiohydrolase from Thermoascus aurantiacus, endoglucanase from Acremonium thermophilum, β-glucosidase from T. auriantiacus and xylanase proteins.

Tabka et al. (2006) describe improved conditions of use of fungal lignocellulolytic enzymes for conversion of lignocellulosic biomass to fermentable sugars for the production of bioethanol. Wheat straw was pretreated with diluted sulfuric acid followed by steam explosion. As synergistic affect between enzymes originating from different fungi was observed: cellulases, xylanase from Trichoderma reesei and feruloyl esterase from Aspergillus niger under a critical enzyme concentration (10 U/g of cellulases, 3 U/g of xylanase and 10 U/g of feruloyl esterase. The yield of enzymatic hydrolysis was enhanced by increasing the temperature from 37° C. to 50° C.

There is a continuous need for new methods of degrading cellulosic substrates, in particular lignocellulosic substrates, and for new enzymes and enzyme mixtures, which enhance the efficiency of the degradation. There is also a need for processes and enzymes, which work at low temperatures, enabling the use of high biomass consistency and leading to high sugar and ethanol concentrations. This approach may lead to significant saving in energy and investments costs. The present invention aims to meet at least part of these needs.

The present invention deals with a method for treating biomass with at least an enzyme mixture obtained from a unique non genetically modified Penicillium funiculosum deposited under Budapest treaty in the

International Mycological Institute under the number IMI 378536, comprising the step of providing biomass and then contacting it with the enzyme mixture as described above under conditions wherein the saccharification of the biomass occurs.

According to the present invention, the enzyme mixture, obtained from a single fungus Penicillium funiculosum deposited under Budapest treaty in the International Mycological Institute under the number IMI 378536 is described in the European patent application No. EP 1 007 743, whose content is incorporated by reference.

According to the description of EP 1 007 743 the above cited Penicillium funiculosum is manufactured by fermentation of the deposited strain first on a seed medium (preferably constituted of (in weight): corn steep liquor 1% to 4%, antifoam just to avoid foam, water to 100%, NaOH enough to adjust the pH to about pH 3.0 to 6.0 before sterilisation of the medium) at a temperature of incubation of 27° C. to 36° C.

The production medium which has preferably the following constitution (in weight): corn steep liquor 0 to 4.0%, batched and fed cellulose 0.8 to 14%, calcium salt: 0 to 0.8%, ammonium sulfate 0 to 1.0%, antifoam just to avoid foam, water enough to obtain 100%, NaOH enough to adjust the pH to about pH 3.0 to 6.0 before sterilisation of the medium; and H2SO4 enough to maintain the pH to about 3.0 to 6.0, ammonia as gas or liquid enough to maintain the pH to about pH 3.0 to 6.0; is used at a temperature of incubation of 27 ° C. to 36 ° C.

The main source of carbon which is added during the process of fermentation is cellulose; amongst different cellulose sources we prefer to use ARBOCEL, SOLKAFLOC, CLAROCEL, ALPHACEL, or FIBRACEL with different grades.

The pH during the fermentation is preferably controlled by the addititon of sulphuric acid, or another acid, and ammonia in gas or liquid form, or another base.

At the end of the fermentation time, solids are eliminated by solid-liquid separation such as filtration or centrifugation, the liquid phase is collected and concentrated for example by ultra-filtration on organic or mineral membrane.

In accordance with the invention, the enzyme mixture may be provided as an isolated pure enzyme preparation or as a crude preparation such as the cultivation medium in which Penicillium funiculosum has been grown. The pure enzyme preparation is commercialized under the trade name Rovabio™ LC. The enzyme mixture of the present invention can be supplemented with additional pure of crude enzyme preparation(s) such as xylanase.

The biomass according to the present invention refers to living and recently dead biological vegetal material that can be used as fuel or for its industrial production. The biomass is composed of both carbohydrate and non-carbohydrate materials. The carbohydrates can be sub-divided into cellulose, a linear polymer of β-1,4 linked glucose moieties, and hemicellulose, a complex branched polymer consisting of a main chain of β-1,4 linked xylose with branches of arabinose, galactose, mannose and glucuronic acids. On occasion the xylose may be acetylated and arabinose may contain ferulic or cinnamic acid esters to other hemicellulose chains or to lignin. The last major constituent of biomass is lignin, a highly cross-linked phenylpropanoid structure.

The method of the present invention can be practiced with the major components of a lignocellulosic biomass, or any composition comprising cellulose (lignocellulosic biomass also comprises lignin), e.g., seeds, grains, tubers, plant waste or byproducts of food processing or industrial processing (e.g., stalks), corn (including cobs, stover, and the like), grasses, wood (including wood chips, processing waste), paper, pulp, recycled paper (e.g., newspaper). In a particular aspect, enzymes of the invention are used to hydrolyze cellulose comprising a linear chain of β1,4-linked glucose moieties. But in preferred embodiments wheat straw, wheat bran, hemp fibers or stalk of peeled hemp are used as biomass.

The processed biomass according to the invention comprises an enzyme mixture obtained from Penicillium funiculosum deposited under Budapest treaty in the International Mycological Institute under the number IMI 378536 wherein the enzyme mixture contains at least xylanases, β-glucanases, cellobiohydrolase, β-glucosidase, cellulases, pectinases and feruloylesterases and can be obtained according to the above described method.

In a further aspect, the method according to the invention can comprise a pretreatment step before contacting with at least the enzyme mixture with the biomass. This pretreatment step aims at increasing the surface area and the accessibility of the biomass to the enzyme

The pretreatment step can of chemical nature such as putting the biomass into a sulfuric acid bath at 98° C., the sulfuric acid being present in a concentration of 3 g/liter or of mechanical nature such as crushing the biomass.

In a still further aspect the invention deals with a method for producing bioethanol comprising the steps of providing at least an enzyme mixture obtained from Penicillium funiculosum deposited under Budapest treaty in the International Mycological Institute under the number IMI 378536, providing biomass, contacting the enzyme mixture and the biomass under conditions wherein the saccharification of the biomass occurs and fermenting the product of said saccharification. In a particular embodiment of the invention, the liquid fraction is isolated pursuant the saccharification, said isolation can be made by centrifugation of the medium comprising the biomass and the enzyme mixture.

The enzyme mixture obtained from Penicillium funiculosum deposited under Budapest treaty in the International Mycological Institute under the number IMI 378536 can be used for the saccharification of biomass.

### FIGURES

FIG. 1: Digestibility of cellulose with Rovabio™ LC

A. The hydrolysis is carried out with 100 μl of Rovabio™ LC/g of substrate, at 37° C. and over times of 24, 48, 72 and 96 h. The percentage of cellulose hydrolysed is determined by weighing dry mass.
B. The amounts of sugar are determined by HPLC analysis of the hydrolysis supernatants. This graph represents the amounts of the various sugars released during the hydrolysis of cellulose.

FIG. 2: Hydrolysis of various lignocellulosic substrates under mild conditions

A. The hydrolysis of the wheat straw and of the wheat bran is carried out with 100 μl of Rovabio™/g of substrate. The incubation is carried out at 28° C. for 72 h.
B. This graph shows that glucose is released from all the substrates and mainly from wheat bran (0.094 g/g of initial S).

FIG. 3: Effect of enzyme concentration on the hydrolysis of wheat straw and of wheat bran

The hydrolysis is carried out for 72 h and at 28° C.
A. Change in hydrolysis of wheat straw and of wheat bran as a function of the enzyme concentration.
B. Amount of Glucose released as a function of the enzyme concentration.

FIG. 4: Amount of glucose released from wheat bran as a function of Rovabio™ LC concentration and of hydrolysis time The maximum amount of glucose released is 0.094 g/g of Sᵢ, this being through the action of 100 μl of Rovabio™ LC/g of substrate for 72 h at the temperature of 28° C.

FIG. 5: Effect of temperature on the hydrolysis of wheat bran

The hydrolysis of the wheat bran is carried out with 100 μl of Rovabio™ LC/g of substrate.
A. Change in hydrolysis of the biomass as a function of temperature and of time.
B. Change in glucose release as a function of temperature and of duration of hydrolysis.

FIG. 6: Effect of the pretreatment with dilute acid on the hydrolysis of wheat straw and of wheat bran

The substrates are incubated for 20 min at 98° C. in 50 ml of 3% sulphuric acid, and then rinsed with 100 ml of ultrapure water. The hydrolysis is carried out at 37° C. with 100 μl of Rovabio™ LC/g of substrate for the two substrates (A). The pretreatment promotes hydrolysis of the substrates but has the opposite effect on the release of glucose (B).

FIG. 7: Hydrolysis of wheat bran with xylanases

P. funiculosum xylanase B is expressed in Pichia pastoris and xylanase C in Yarrowia lipolytica. The xylanase concentrations are such that the xylanase activity is equivalent to that contained in 200 μl of Rovabio™ LC. Roy. -Xyn B is a mixture in which 50% of xylanase activity comes from Rovabio™ LC and the other half is provided by xylanase B (idem for the Rov. -Xyn C mixture, where Xyn C is xylanase C). The wheat bran hydrolysis is carried out at 37° C. for 72 h.
A. Comparison of the hydrolysis of wheat bran with various enzymes.
B. Xylose and glucose released from wheat bran with various enzymes.

### EXAMPLES

### Example 1

### Materials and Methods

1. Enzymes and Substrates

Rovabio™ LC is the cocktail of enzymes obtained from Penicillium funiculosum deposited under Budapest treaty in the International Mycological

Institute under the number IMI 378536 of which the main known activities are cellulase, xylanase and β-glucanase.

P. funiculosum xylanases were also tested. These are P. funiculosum xylanase B cloned in Pichia pastoris and P. funiculosum xylanase C cloned in Yarrowia lipolytica.

P. funiculosum fermentation musts were also used. This is because Rovabio™ LC is a formulated product, and we therefore wanted to test the hydrolytic capacity of a crude enzyme cocktail.

The substrates selected are: first-quality wheat straw (Val Agro, batch 37600205113), wheat bran (unknown origin), wheat straw (unknown origin) and cellulose (JRS, Arbocel batch 16 006 80 121).

2. Preparation of Substrates

For two of the substrates studied, a mixing step is necessary in order to optimize the accessibility of the enzymes to the substrate. They are the first-quality wheat straw and the wheat straw. They were reduced using a mixer, the final particle size being of the order of a centimetre.

For the subsequent steps, all the substrates are treated identically. The substrate is weighed out into an Erlenmeyer flask, and then 50 ml of 0.1 M acetate buffer, pH 5.4, are added. The Erlenmeyer flasks are subsequently autoclaved for 20 min at 121° C.

3. Chemical Pretreatment

In parallel to tests carried out under mild conditions, the wheat straw and the wheat bran was subjected to a chemical pretreatment. The aim of the pretreatment is to alter the physical structure of the biomass and to separate the various fractions in order to make the cellulose more accessible to the enzymes, which will then be able to convert it to fermentable sugars. These substrates are brought into contact with 20 ml of sulphuric acid at 3 g/l and then incubated in a water bath for 20 min at 98° C. They are subsequently rinsed with 100 ml of ultrapure water. The rinsing water is removed and the pretreated substrates are subsequently treated in the same manner as the other substrates, namely addition of acetate buffer and then autoclaving.

4. Enzymatic Hydrolysis

After the Erlenmeyer flasks have been cooled to ambient temperature, the enzyme solution is added to them under sterile conditions. Each condition is tested in duplicate and a control is integrated into each test. The control comprises an Erlenmeyer flask to which the enzyme is not added. Various concentrations of enzyme were tested in order to determine the amount of enzyme/amount of substrate ratio that is the most effective, i.e. the greatest possible ratio. For the tests involving enzymes other than Rovabio™ LC, the amounts used were determined such that the cellulase and/or xylanase activities are equivalent to those of Rovabio™ LC.

The Erlenmeyer flasks are subsequently incubated for 24, 48 or 72 h, with stirring at 150 rpm and at a given temperature. During the various tests, the temperatures tested are 28, 30 and 37° C.

5. Separation of Soluble and Insoluble Fractions

After incubation, the insoluble fraction of the various samples is recovered by centrifugation at 10 000 g for 15 min at 4° C. The supernatant is aliquoted and stored at −20° C. in order to be subsequently analyzed by HPLC. As regards the pellet, it is washed for a first time with 50 ml of water and then again with 40 ml of water. After each wash, the pellet is recovered by centrifugation (same conditions as previously) and the washing supernatants are also aliquoted in order to be analysed by HPLC.

In addition, in order to estimate the percentage of substrate converted to soluble sugars, the pellet is dried at 120° C. for 24 h and then weighed. The percentage of insoluble biomass remaining after hydrolysis is equal to the ratio: (remaining dry mass/initial dry mass)×100. In order to determine the initial dry masses, independently of the hydrolysis tests, each substrate was weighed and then dried for 24 h at 121° C. Thus, the difference in mass between the fresh substrate and the dry enables us to determine the percentage of moisture contained in the biomasses. It is subsequently sufficient to apply the percentage of moisture to each weighing of fresh substrate in order to determine its dry mass.

6. Analysis of Supernatants by HPLC

In order to determine the sugar composition of the soluble fraction, the centrifugation supernatants and also the washing supernatants are analysed by HPLC (Agilent 1100). The chromatographic system comprises an isocratic pumping system, a sample changer, a precolumn (Bio-Rad, Micro-Guard® Carbo P), an Aminex® HPX-87P column (Bio-Rad), an RID (Refractive Index Detection) detector or refractometer and a data acquisition and processing system. Before being injected into the chromatography column, the supernatants are centrifuged at 16 100 g for 30 min and at 4° C. in order to pellet any impurities. They are subsequently filtered through a 0.45 μm cellulose membrane. The analytical conditions are the following: the chromatography is carried out at a temperature of 80° C., the mobile phase is ultrapure water, the flow rate is 0.6 ml/min, and 20 μl of sample are injected, followed by washing with 100 μl of water. The constituents of the sample are identified by their retention time by means of a pre-established calibration range. This range is composed of 4 sugars, the concentration of which ranges from 0.5 g/l to 25 g/l. The sugars used for the calibration range are cellobiose, D-glucose, D-xylose and L-arabinose. They are sugars predominantly released during the hydrolysis of lignocellulosic biomasses.

7. Assaying of Cellulase and Xylanase Activities

The enzyme activities are measured by means of the 3,5-dinitrosalicylic acid (DNS) assay method (G. L. Miller, 1959). The unit of cellulase or of xylanase activity is defined as the amount of enzyme required for the release of one μmol of glucose equivalent or xylose equivalent, respectively, per minute and per gram of product under the enzymatic conditions defined, namely pH 5 for the cellulase activity and pH 4 for the xylanase, and at a temperature of 50° C.

For the cellulase activity, the test is based on the enzymatic hydrolysis of carboxymethylcellulose (CMC), which is a polymer of glucoses connected by β-1,4 linkages. The enzymatic hydrolysis releases glucose monomers, the concentration of which is determined at the end of the reaction by colorimetric assay and using a standard curve for glucose, the absorbance of which is measured at 540 nm. The enzyme dilutions are made in ultrapure water. Each sample is assayed in duplicate in order to obtain an average activity and a control is also prepared. 1.75 ml of substrate (1.5% w/v CMC) are placed in test tubes and then incubated in a water bath for 5 min at 50° C. The reaction is then initiated by adding 250 μl of enzyme dilution, except in the control tubes. It is then stopped by adding 2 ml of 1% (w/v) of DNS after exactly 10 min, still at 50° C. At this stage, the tubes are removed from the water bath, 250 μl of enzyme dilution are added to the control tubes, and then all the tubes are stoppered and transferred into a second water bath at 95° C. for exactly 5 min. This step allows the DNS (orangey coloration) to be reduced, by the released glucose, to 3-amino-5-nitrosalicylic acid (orangey-red coloration). The tubes are subsequently placed in a bath of cold water in order to return to ambient temperature. Finally, an additional dilution is carried out by adding 10 ml of water, and the absorbance can then be read at 540 nm.

For the xylanase activity, the principle of the assay remains the same. Since the substrate is birch wood xylan at 1.5% (w/v), the enzymatic hydrolysis releases xylose monomers which have the same reducing role as the glucose for the cellulase activity. The standard range on the other hand is prepared with xylose.

### Example 2

### Evaluation of the Conversion of a Simple Substrate by Rovabio™ LC: Cellulose

The commercial cellulose that we use is in reality a mixture of true cellulose and of hemicellulose. Cellulose is a polymer of β-1,4-linked D-glucoses. Rovabio™ LC, by virtue of its cellulase activities (endo-1,4-β-glucanase, cellobiohydrolase and β-glucosidase), would therefore hydrolyse it and release glucose monomers, on the basis of the synthesis of bioethanol. Hemicellulose is a polymer of D-xyloses which are also β-1,4-linked, and is branched with various sugars, such as mannose, galactose, arabinose, etc. It is also hydrolyzed by Rovabio™ LC by virtue of the xylanase activities of the latter (endo-1,4-β-xylanase, β-xylosidase, α-arabinofuranosidase, among others).

Since the cellulose is 99.5% pure (according to the supplier), we can thus estimate the maximum hydrolytic capacity of Rovabio™ LC (mainly its cellulase activity and also the xylanase activity) under experimental conditions which are closer to biomass conversion tests rather than enzyme activity assays. In order to estimate the yield for conversion of the cellulose into soluble sugars, we use two methods as a basis. The first consists of weighing dry masses; the second enables us to determine the amount and the nature of the sugars released by means of HPLC analysis of the hydrolysis and washing supernatants.

For this substrate, the enzymatic hydrolysis is carried out at a Rovabio™ LC concentration of 100 μl/g of substrate and at a temperature of 37° C. It is monitored between 24 and 96 h of incubation.

1. Dry Masses

The hydrolysis yield is estimated by the percentage of dry biomass remaining after reaction.

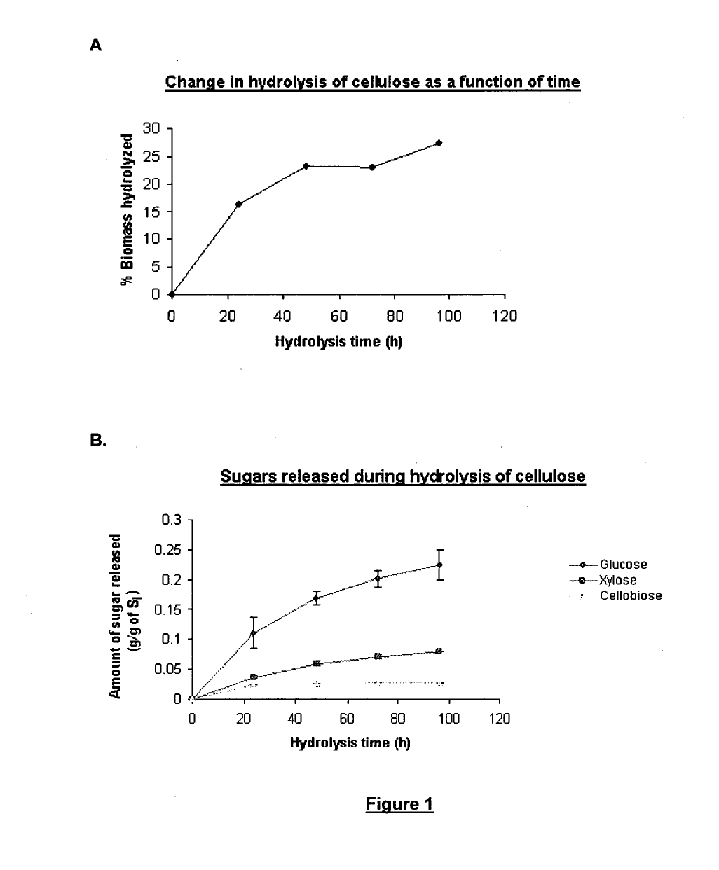

Since cellulose is a simple polymer, unlike the other biomasses studied, its hydrolysis should be at a maximum, i.e. should be nearing the 90-99% range, and very certainly in a relatively short period of time, as has been observed for the enzyme complex Accelerase™ 1000 (technical bulletin No. 1, Genencor). However, as shown in FIG. 1.A, the cellulose hydrolysis reaches only 27.3% after 96 h. In fact, after hydrolysis, approximately 70% of insoluble fraction is recovered. This result is surprising in view of the hydrolysis conditions and of the nature of the substrate. One explanation for this result could be hydrolysis conditions that are too mild (temperature, pH, etc.), or too low a concentration of enzyme relative to the amount of substrate, or else, on the contrary, an inhibition of certain cellulases by the cellobiose released by cellobiohydrolases (Väljamäe P. et al., 2001). In order to verify this hypothesis, the results of the HPLC analysis of the supernatants should be examined.

2. Analysis of Release Sugars

Since the cellulose is 99.5% pure, we can expect the 28% hydrolysed to correspond to glucose, cellobiose in a lesser amount and xylose originating from the hemicellulosic fraction. FIG. 1.B shows the various sugars released during the cellulose hydrolysis. The amount of xylose released reaches 0.08 g/g of initial solids (Sᵢ), i.e. 8 g released from 100 g of cellulose.

As regards the release of glucose, the release of 0.225 g/g of Sᵢ is obtained after 96 h. This result is in agreement with the difference in dry mass mentioned above. In fact, the hydrolysis solubilized 28% of cellulose which is found in the form of glucose (22.5%) and xylose (8%). Logically, the amount of glucose released from the cellulose should represent the maximum amount that can be released during hydrolysis of Iignocellulosic biomass with Rovabio™ LC.

Finally, the analysis of the supernatants also reveals the presence of cellobiose. The cellobiose is released from the cellulose by virtue of the cellobiohydrolase activity of our enzyme cocktail. Its concentration is constant and relatively low between 24 and 96 h (approximately 0.026 g/g of Sᵢ). There was therefore no inhibition by the substrate, otherwise the cellobiose concentration would increase with the hydrolysis time and the glucose concentration would remain constant. The low level of cellulose hydrolysis is therefore probably due to an insufficient concentration of enzyme or to the hydrolysis conditions being too mild. However, we chose to carry out tests under relatively mild conditions compared with those performed industrially at the current time. This choice is guided by the need to show the effectiveness of Rovabio™ LC under less expensive conditions. The subsequent tests on more complex biomasses would therefore be carried out under the conditions initially set.

### Example 3

### Hydrolysis of Various Biomasses Under Mild Conditions

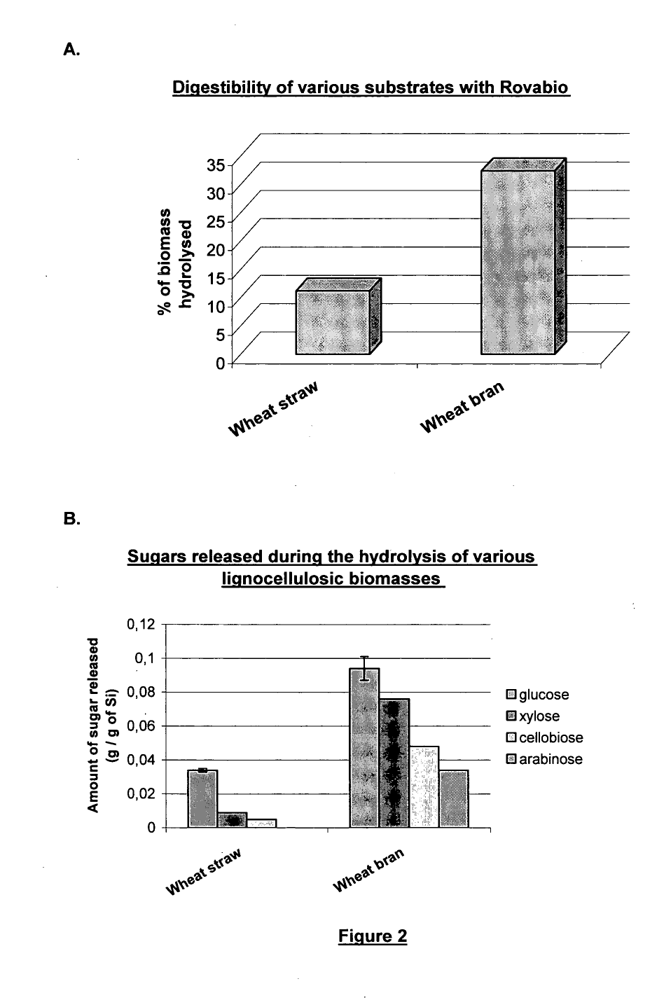

For the following tests, the amount of substrate tested is 2 g, the concentration of Rovabio™ LC is 100 μl/g of substrate, and the hydrolysis conditions are the following: 28° C. for 72 h. The biomass hydrolysis results are represented in FIG. 2 A, the HPLC analysis results in FIG. 2 B.

1. Wheat

Wheat is one of the substrates most widely used in the production of bioethanol in France. It is also the substrate which has given the best results for Rovabio™ LC hydrolysis yield. We studied it in two different forms: wheat straw and wheat bran.

1.1. Wheat Straw

It is composed of 33 to 43% of cellulose, 20 to 25% of hemicellulose and between 15 and 20% of lignin (source IFP, 2007). Since the size of the strands was too great, it was reduced by mixing. The final particle size is of the order of a centimeter.

After enzymatic hydrolysis, the amount of insoluble biomass recovered is 80%, that recovered for the controls is 90%. The hydrolysis therefore produced a decrease in biomass of 11.2%.

As regards the HPLC analysis of the supernatants, the release of the four sugars used in our calibration range is observed for this substrate. A small amount (0.005 g/g of Sᵢ) of cellobiose is present, which suggests that there may be efficient hydrolysis by β-glucosidases. Arabinose is also detected, but in trace form, likewise xylose is released at a concentration of 0.009 g/g Sᵢ. The presence of these two sugars is characteristic of the hemicellulase activities of Rovabio™ LC (endo-1,4-β-xylanase, α-arabinofuranosidase and β-xylosidase, in particular). The amount of glucose released during the hydrolysis is 0.034 g/g of Sᵢ. Even though it is the sugar predominantly released under these hydrolysis conditions, its concentration remains too low to envisage wheat straw as base substrate in bioethanol synthesis.

Straw, despite being mixed, remains a very raw material. However, enzymatic hydrolysis is promoted by the use of substrates which have a small surface and a low proportion of lignin and in which the crystallinity of the cellulose is low. Wheat bran is a substrate which has these characteristics.

1.2. Wheat Bran

It is difficult to accurately determine the proportions of each constituent of wheat bran, since this composition differs according to the origin of the wheat and to the milling of said wheat, and also according to the method of analysis used. However, it contains on average between 3 and 7% of lignin (Schwartz et al., 1988) and its soluble sugar composition appears to be the following: 2.1% of galactose, 23.7% of arabinose, 29.1% of glucose and 43.7% of xylose (Benamrouche et al., 2002).

Over the course of our tests, wheat bran is the substrate which showed the best propensity for enzymatic hydrolysis. Specifically, a decrease in biomass of 32.5% is observed after hydrolysis. Moreover, the HPLC analysis of the supernatants revealed that the soluble fraction is composed of 0.048 g of cellobiose/g of Sᵢ; 0.034 g of arabinose/g of Sᵢ; 0.076 g of xylose/g of Sᵢ and 0.094 g of glucose/g of Sᵢ. Thus, under mild hydrolysis conditions, approximately 10% of the amount of substrate hydrolysed was released in the form of glucose. The proportions of soluble sugars that we obtained were not similar to those predicted by Benamrouche et al., because, on the one hand, the enzymatic hydrolysis is probably not total and, on the other hand, the sugar composition of the wheat bran can vary significantly with the origin and the milling of the latter.

Wheat bran appears to be an ideal substrate for the release of glucose.

These first tests were carried out under relatively mild hydrolysis conditions (28° C., for 72 h and an enzyme concentration of 100 μl/g of substrate).

### Example 4

### Optimization of the Hydrolysis Conditions

This optimization involves 3 essential factors: the enzyme concentration, the hydrolysis temperature and the chemical pretreatment in order to improve the accessibility of the substrate to the enzymes.

1. Effect of the Enzyme Concentration

In order to increase the release of glucose from our substrates, the logical reasoning would be to increase the enzyme concentration. However, to date, the main obstacle in the development of biofuels is the cost of the enzymes used in the saccharification step for lignocellulosic biomasses. For this reason, it is necessary to define the optimum enzyme concentration.

We therefore tested the effects of the enzyme concentration on two of our substrates: wheat straw and wheat bran, which, under the previous conditions, gave the best results. Other than the amount of enzyme used, the analysis conditions are unchanged, i.e. an incubation at 28° C. for 72 h and with stirring at 150 rpm. We are mainly interested in the percentage of biomass hydrolysed and in the amount of glucose released.

For the wheat straw, three concentrations were evaluated: 40, 100 and 200 μl of Rovabio™ LC/g of substrate. In view of the results obtained for a concentration of 100 μl of Rovabio™ LC/g of straw, it was decided to test a higher concentration despite the cost of the treatment.

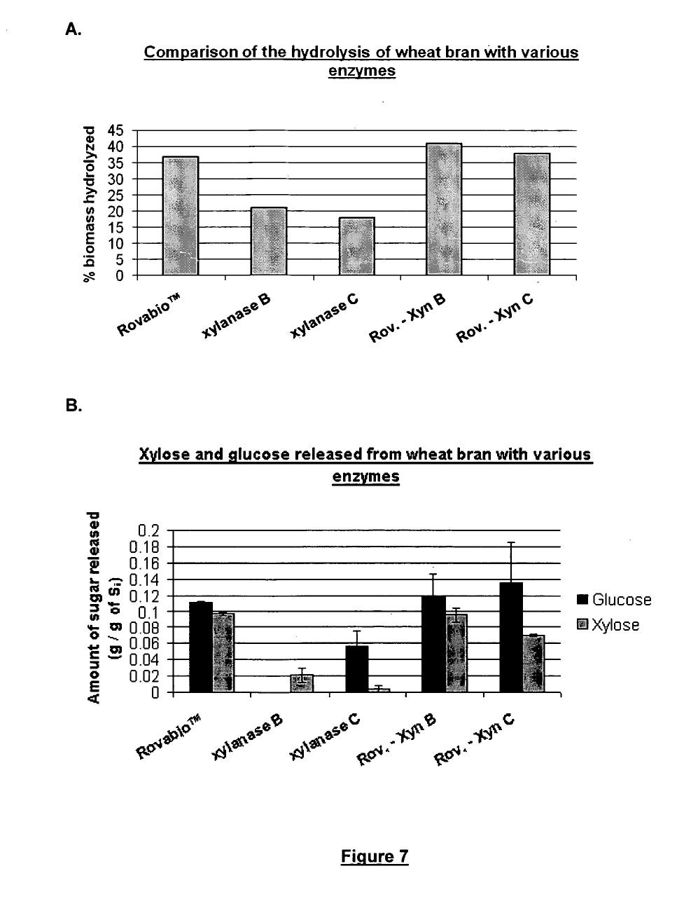

As regards the effect of the hydrolysis on the dry biomass, a 1.2%, 11.2% and 15% decrease in the latter is observed for the concentrations of 40, 100 and 200 μl of Rovabio™ LC/g of substrate, respectively (FIG. 7 A). It should be noted that doubling the concentration of Rovabio™ LC does not lead to hydrolysis of the wheat straw in the same proportions.

As expected, the greatest release of glucose is observed for the enzyme concentration of 200 μ/g of substrate. Specifically, for the increasing Rovabio™ concentrations, 0.025; 0.034 and 0.067 g of glucose/g of Sᵢ, respectively, are obtained (FIG. 7 B). In this case, the increase in released glucose follows the increase in the enzyme concentration. It is advantageous to note that, for a concentration of 40 μl of Rovabio™ LC/g of substrate, the equivalent of 73% of glucose released per 100 μl of Rovabio™ LC/g of substrate is released. This result brings to the fore the possibility of using less enzyme while at the same time keeping the effectiveness of the cellulases. The hydrolysis also enabled the release of xylose in virtually identical amounts for the concentrations of 40 and 100 μl/g of substrate (0.007 and 0.009 g/g of Sᵢ, respectively) and 0.018 g of xylose/g of Sᵢ for 200 μl of Rovabio™/g of substrate.

For the wheat bran, we studied four different concentrations: 20, 40, 50 and 100 μl of Rovabio™ LC/g of substrate.

The increase in Rovabio™ LC concentration has the effect of increasing the hydrolysis of the wheat bran, but as for the wheat straw, not proportionally. In fact, in increasing order of concentration, the following decreases in dry biomass are obtained: 23, 25, 32 and 32.5% of wheat bran hydrolyzed. 50 μl of Rovabio™ LC/g of substrate are therefore sufficient to achieve maximum hydrolysis. It would therefore appear that, for the hydrolysis conditions which were used, i.e. at pH 5.4 and for a temperature of 28° C., the maximum hydrolysis of the substrates with Rovabio™ LC does not exceed approximately 30%.

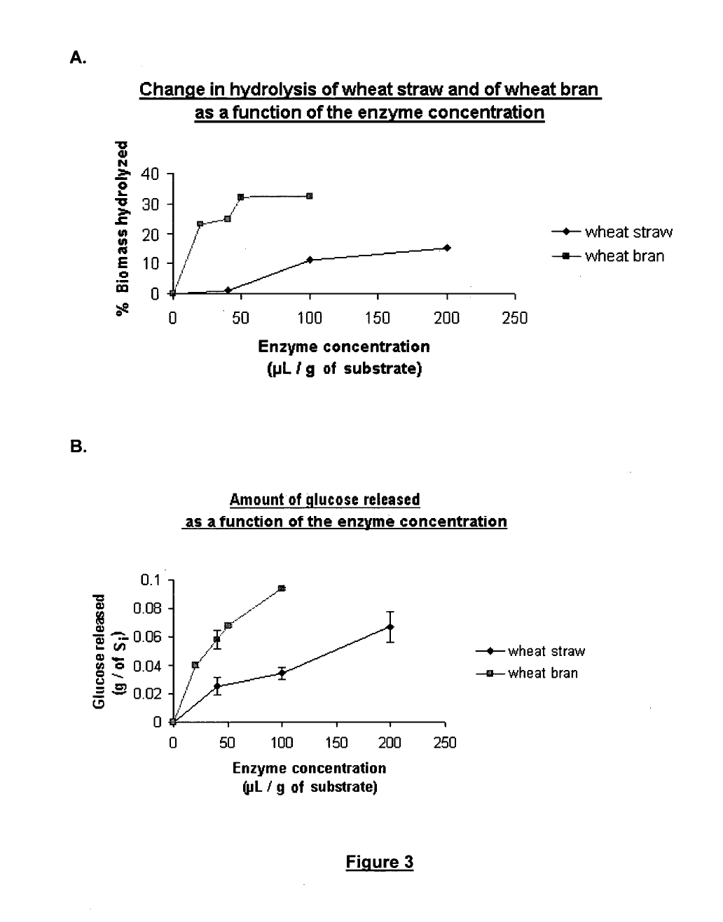

As regards the HPLC analysis of the supernatants, it also reveals the advantage of using higher enzyme concentrations. In fact, the amount of glucose released increases with that of the Rovabio™ LC used to carry out the hydrolysis. The amounts released are the following: 0.04; 0.058; 0.068 and 0.094 g of glucose/g of Sᵢ for the respective Rovabio™ LC concentrations of 20, 40, 50 and 100 μl/g of substrate (FIG. 3). It is important to note that, while the maximum amount of glucose is obtained for the highest Rovabio™ LC concentration, an increase in enzyme concentration of 50% results in an increase in glucose released of only 28%. It is therefore possible to reduce the amount of enzyme used without, however, excessively decreasing the amount of glucose released.

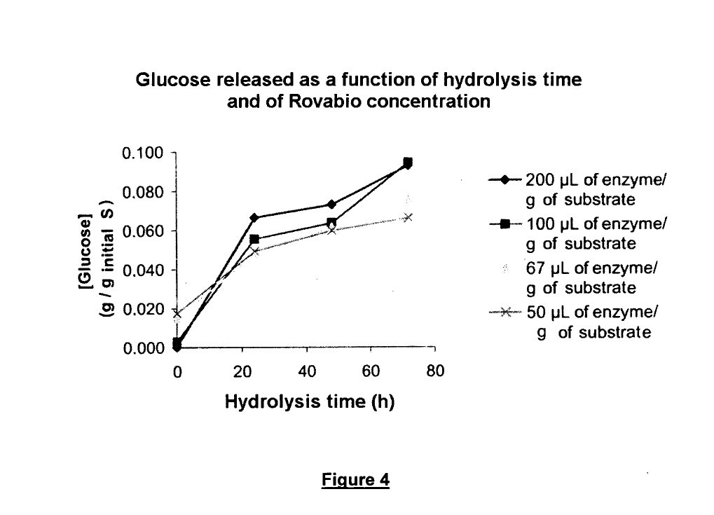

Furthermore, we also analyzed the supernatants from hydrolyses carried out with various concentrations of Rovabio™ LC and which lasted 24, 48 and 72 h (FIG. 4). Several conclusions then emerged. First of all, for the same hydrolysis time (24, 48 or 72 h) the maximum amount of glucose is always released by the highest enzyme concentration. In addition, for the same Rovabio™ LC concentration, the maximum amount of glucose released is obtained after 72 h. However, from 48 h onwards, the appearance of a plateau corresponding to 80% of the maximum amount of releasable glucose was observed. The same release profile is observed for xylose, with a maximum concentration of 0.076 g/g of Sᵢ obtained for 72 h of hydrolysis and a Rovabio™ LC concentration of 100 μl/g of substrate.

All these results clearly confirm the fact that a hydrolysis can be carried out either with a lower concentration of Rovabio™ LC and over a longer hydrolysis time, or with a high enzyme concentration but over a shorter hydrolysis time.

2. Effect of Temperature

The previous tests were carried out at a temperature of 28° C., but this is not the optimum temperature for Rovabio™ LC activity. In fact, this enzyme cocktail is composed of various activities which are normally assayed at a temperature of 50° C. Moreover, Rovabio™ LC is first and foremost a nutritional additive used in animal feed. It must therefore be possible for it to be active at the temperature of the digestive tract of the animals, which varies between 38 and 41° C. depending on the species. Tests on hydrolysis of lignocellulosic biomasses therefore had to be carried out at temperatures above 28° C.

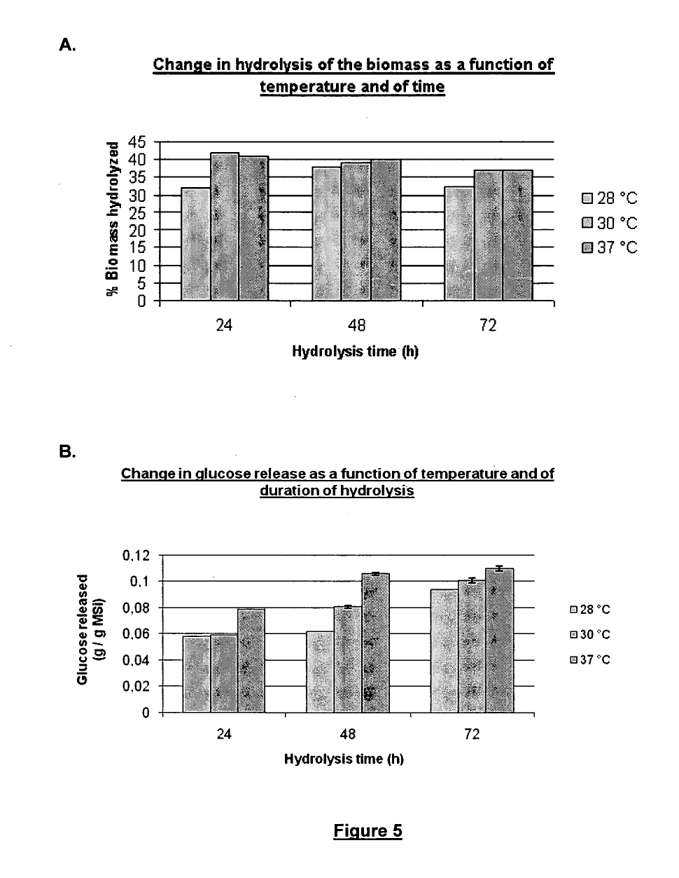

In order to estimate the effect of the temperature on the hydrolysis of lignocellulosic biomasses with Rovabio™ LC, we carried out a series of tests with wheat bran as substrate, in a Rovabio™ LC concentration of 100 μl/g of substrate, over 24, 48 and 72 h and at 3 different temperatures: 28, 30 or 37° C. (FIG. 5A). The substrate and the enzyme concentration were determined according to the best results obtained in the previous tests. When the percentages of biomass hydrolyzed in this test are observed, several surprising points are noted (FIG. 5B). Firstly, there is only a slight difference between hydrolysis at 30 and at 37° C., irrespective of the hydrolysis time. On the other hand, if they are compared to the hydrolysis carried out at 28° C., a slight increase in the percentage of biomass hydrolyzed is noted (on average, 32%, 39% and 39% of wheat bran are hydrolyzed at the respective temperatures of 28, 30 and 37° C.). It is therefore clear that the hydrolysis is promoted by temperatures above 28° C.

The effect of the temperature on the various enzyme activities, and more specifically on the cellulase activity, will now be observed.

Unlike the results obtained by treatment of the dry biomasses, the results from analyzing the supernatants are more coherent. In fact, the amount of glucose released increases over time and reaches a maximum in the tests carried out at 37° C. Specifically, the maximum concentration obtained is 0.11 g of glucose/g of Sᵢ for 72 h of hydrolysis at 37° C. In this test, an increase in the temperature of approximately 10° C. therefore makes it possible to increase the amount of glucose released by 14.5%.

As regards xylose, the release profile is similar to that of glucose and the maximum amount of xylose released is 0.096 g/g of Sᵢ. This is of value since xylose can also be recovered in the bioethanol production process.

Even though the temperatures tested are very similar, an increase in the amount of soluble sugars released can be noted in parallel with the increase in hydrolysis temperature. Since the temperature used in the Rovabio™ LC activity assays is 50° C., it is therefore possible to envisage optimizing the hydrolysis of lignocellulosic biomasses again, at temperatures above 37° C.

Despite the need to define less expensive hydrolysis conditions, in certain cases, the addition of steps to the saccharification process is inevitable, in particular when the substrates have a hemicellulose or lignin composition that is too high.

3. Effect of the Chemical Pretreatment

The substrates used for the production of bioethanol are materials that are relatively raw and may require a pretreatment before the enzymatic hydrolysis. Various types of pretreatment exist, and the aim of all of them is to improve the accessibility of the substrate to the enzymes by reducing the size of the particles or by reducing the hemicellulose and/or lignin fraction. Many pretreatments have been developed: physical pretreatments (substrate pressurized), heat pretreatments (steam explosion) (Mosier N. et al., 2005) or else chemical (acidic or basic) pretreatments. The latter are by far the most widely used, not only on the laboratory scale, but also at the industrial development stage (Schell D. J. et al., 2003).

The one we chose to carry out is a pretreatment with 3% sulphuric acid at a temperature of 98° C. These pretreatment conditions differ from those customarily used. In fact, the pretreatments are carried out at higher temperatures (from 100 to 200° C.) but at low acid concentrations (approximately six times less concentrated) (Lloyd T. A. et al., 2005; Wyman C. E. et al., 2005). We have previously seen that wheat straw have a high percentage of hemicellulose and of lignin, which are protective layers for the cellulose. The acid pretreatment has the property of removing the hemicellulosic fraction and of altering the structure of the lignin, thus making the cellulose accessible to enzymes. We compared the effectiveness of the acid pretreatment on two of the substrates studied above wheat straw, which did not give significant hydrolysis under mild conditions, and wheat bran.

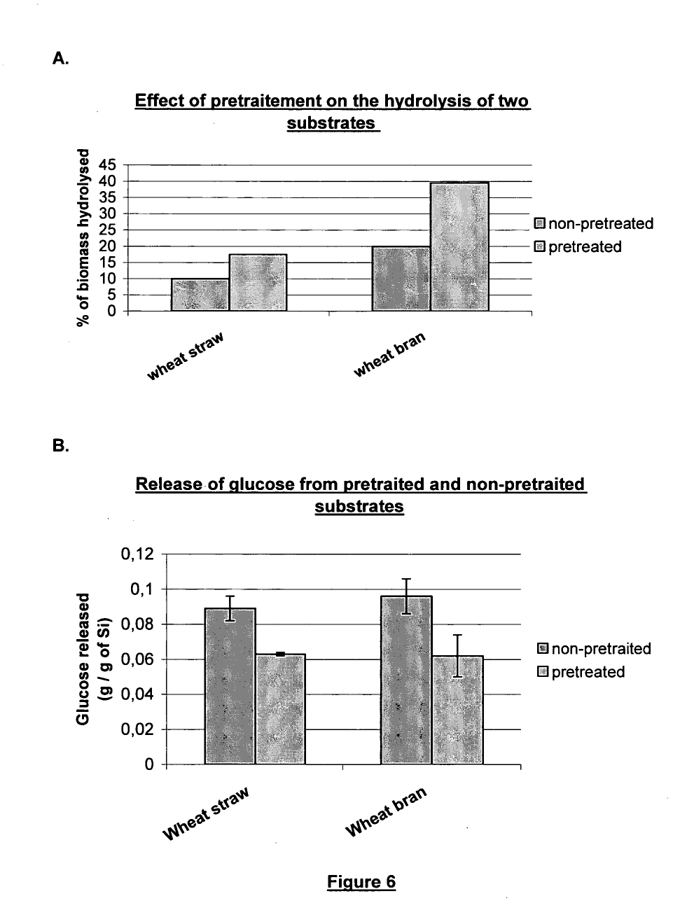

The percentages of biomass pretreated and then hydrolysed with Rovabio™ LC are the following: 17.5% of wheat straw hydrolysed against 10% without pretreatment, and 39.6% of wheat bran hydrolysed whereas, without pretreatment, the hydrolysis reaches 20% (FIG. 6 A). The pretreatment therefore appears to have a positive effect on the hydrolysis of the substrates with Rovabio™ LC.

On the other hand, when the amount of glucose released is addressed, the effectiveness of the pretreatment is less obvious (FIG. 6 B). For the wheat straw, a concentration of 0.063 g of released glucose/g of Sᵢ is obtained, i.e. a loss of 30% compared with a hydrolysis without pretreatment. Finally, for the wheat bran, the amount of released glucose is 0.062 g/g of Sᵢ, i.e. 34% less compared with the hydrolysis without pretreatment. Following these results, it is obvious that the pretreatment does not have the expected effect on the release of glucose from these biomasses. Two explanations may be proposed: either the acid pretreatment degraded the cellulose, in which case the glucose would be in the acid solution after heating, or the pH of our substrate is too low after pretreatment and inactivates the cellulase activity of the Rovabio™ LV.

In order to verify these two hypotheses, we firstly analysed, by HPLC, the washing supernatant after pretreatment. The analysis did not reveal the presence of any soluble sugar; the cellulose is not therefore solubilized by the pretreatment. Secondly, we verified the pH of the pretreated substrate in 50 ml of 0.1 M acetate buffer, pH 5.4 (mixture before hydrolysis) and that of a non-pretreated substrate in the same buffer. The pH of the pretreated mixture is 5.1, that of the non-pretreated substrate is 5.6. We therefore assayed the cellulase activity of Rovabio™ LC by the DNS assay method at pH 5.1 and at pH 5.6 in order to verify whether the activity is affected by the decrease in pH (the DNS assay is usually carried out at pH 5). A difference of 7% is observed between the two assay conditions, which does not however explain the effect of the pretreatment on our substrates, firstly because the difference in activity is too small, and secondly because the cellulase activity is greater for a pH lower than that applied to our tests.

However, another explanation is possible: the pretreatment with dilute acid may result in the degradation of the sugars due to a pH which is too acidic (Ogier et al., 1999). These degradations would alter the substrates which would therefore no longer be recognized by the enzymes.

Up until now, we have focused on the properties of Rovabio™ LC in the lignocellulosic biomass saccharification process with the main aim of generating glucose from these biomasses. This is because glucose is the preferred substrate of the organisms used to date to produce bioethanol. However, new, genetically modified organisms capable of assimilating both glucose and xylose as carbon source are beginning to be used. It is therefore advantageous to study the effect of pure xylanases on a lignocellulosic substrate. In addition, bioethanol production must be carried out less expensively. Now, Rovabio™ LC is a formulated product; we are therefore also going to study the ability of a P. funiculosum fermentation must to hydrolyse a lignocellulosic substrate.

### Example 5

### Hydrolysis with Xylanases and the Fermentation Must

The following tests were carried out only on wheat bran. The analysis conditions are the following: hydrolysis at 37° C. (the most effective temperature) for 72 h. The enzyme concentrations are defined such that, for the xylanases, the activity is equivalent to that of 200 μl of Rovabio™ LC and, for the fermentation must, a cellulase activity comparable to that of 200 μl of Rovabio™ is maintained. The results are given in FIG. 7.

1. Xylanase B

The concentration of xylanase B required in order to have an activity equivalent to that of Rovabio™ LC is 150 μl/g of substrate. After hydrolysis with xylanase B, 79% of wheat bran is recovered, which means that the hydrolysis reaches 21%.

Furthermore, the HPLC analysis of the supernatant shows the presence of a single sugar: 0.021 g of xylose/g of Sᵢ, i.e. 4.6 times less than with Rovabio™ LC under the same hydrolysis conditions. This result can be explained by the fact that Rovabio™ LC is an enzyme cocktail composed of various activities which act in synergy with one another, thus facilitating the degradation of complex substrates to soluble sugars.

The gene of another P. funiculosum xylanase was overexpressed: it is xylanase C.

2. Xylanase C

The xylanase C concentration used for these tests is 850 μl/g of substrate. Only 18% of wheat bran is hydrolysed with xylanase C after 72 h of reaction. It would seem that xylanase C is less efficient than xylanase B for hydrolysing this type of substrate.

On the other hand, the results of the HPLC analysis are surprising. Specifically, xylose is found in trace amounts (0.004 g/g of Si) in the hydrolysis supernatant, along with glucose at a concentration of 0.057 g/g of Sᵢ. The amount of xylose released is low despite the effort made to maintain a xylanase activity equivalent to that of Rovabio™ LC. In addition, the presence of glucose is unexpected given that a xylanase is being tested. It would therefore appear that P. funiculosum xylanase C also has a cellulase activity.

Xylanases alone do not have the same ability to release xylose from wheat bran as Rovabio™ LC, probably because the pure xylanases do not benefit from the complementarity of the various activities of Rovabio™ LC.

3. Rovabio™ and Xylanase Synergy

The following tests consist in carrying out the enzymatic hydrolysis of wheat bran with 50% of xylanase activity provided by Rovabio™ LC, the remaining 50% by xylanase B or by xylanase C. The reaction is carried out at 37° C. for 72 h.

The Rovabio™ LC-xylanase B mixture results in the hydrolysis of 41% of the wheat bran, whereas the Rovabio™ LC-xylanase C mixture results in a hydrolysis of 38%. The hydrolysis with Rovabio™ LC gives a wheat bran hydrolysis of 37%. The overall hydrolytic efficiency is therefore maintained; the xylanase here supplements the action of Rovabio™ LC.

Furthermore, the release of 0.12 g of glucose and 0.095 g of xylose/g of Sᵢ is obtained by virtue of the combined action of Rovabio™ LC and of xylanase B. The Rovabio™ LC-xylanase C mixture, for its part, results in the release of 0.136 g of glucose/g of Sᵢ and 0.07 g of xylose/g of Sᵢ. These results are slightly better than those obtained for the hydrolysis of wheat bran or with Rovabio™ LC alone, thereby confirming the importance of the interactions between the various enzymes of the complex that is Rovabio™ LC.

After having studied the hydrolysis of wheat bran with xylanases, one test remains to be carried out in order to complete this study on the enzymatic hydrolysis of lignocellulosic substrates: the hydrolysis of wheat bran with fermentation must.

4. Fermentation Must

We therefore analyzed the hydrolysis of wheat bran with 587 μl of P. funiculosum fermentation must, at 37° C. and for 72 h. The fermentation must that we use for this test corresponds to the fermentation supernatant centrifuged at 1800 g, at 4° C.

A 44% hydrolysis of wheat bran is obtained, which represents a substantially greater hydrolysis than over the course of the other tests. The hydrolysis supernatant gives 0.129 g of glucose/g of Sᵢ and 0.106 g of xylose/g of Sᵢ. This time again, the amounts of glucose and xylose released are slightly greater than those obtained by hydrolysis of the wheat bran with Rovabio™ LC.

Benamrouche S, Cronier D, Debeire P, Chabbert B. (2002). A chemical and histological study on the effect of (1→4)-β-endo-xylanase treatment on wheat bran. Journal of cereal science, 36 (2):253-260.
Lloyd T A, Wyman C E. (2005). Total Sugar Yields for Pretreatment by Hemicellulose Hydrolysis Coupled with Enzymatic Hydrolysis of the Remaining Solids. Bioresource Technology, 96 (18): 1967-1977, 2005.
Miller G. L. (1959). Use of dinitrosalicylic acid reagent for determination of reducing sugar. Analytical Chemistry, vol. 31, p. 426-428.
Mosier N, Wyman C, Dale B, Elander R, Lee Y Y, Holtzapple M, Ladisch M. (2005 a). Features of promising technologies for pretreatment of lignocellulosic biomass. Bioresource technology, 96(6):673-86.
Mosier N, Hendrickson R, Ho N, Sedlak M, Ladisch M R. (2005 b). Optimization of pH controlled liquid hot water pretreatment of corn stover. Bioresource technology, 96(18):1986-93.
Ohgren K, Vehmaanrerä J, Siika-Aho M, Galbe M, Viikari L, Zacchi G (2007). High temperature enzymatic prehydrolysis prior to simultaneous saccharification and fermentation of steam pretreated corn stover for ethanol production. Enzyme and Microbial Technology 40,: 607-13.
Ogier J C, Leygue J P, Ballerini D, Pourquie J and Rigal L. (1999). Production d'éthanol a partir de biomasse ligno-cellulosique. Oil & Gas Science and Technology-Rev. IFP, 54(1):67-94.
Schell D J, Farmer J, Newman M, McMillan J D. (2003). Dilute-sulfuric acid pretreatment of corn stover in pilot-scale reactor: investigation of yields, kinetics, and enzymatic digestibilities of solids. Applied biochemistry and biotechnology, 105 -108:69-85.
Schwarz P B, Kunerth W H, Young V L. (1988). The distribution of lignin and other components within hard red spring wheat bran. Chemical chemistry, 65: 59-64.
Tabka M G, Herpoël-Gimbert I, Monod F, Asther M, Sigoillot J C (2006). Enzymatic saccharification of wheat straw for bioethanol production by combined cellulose, xylanase and feruloyl esterase treatment. Enzyme and Technology 39, 897-902.
Väljamäe P, Pettersson G, Johansson G. (2001). Mechanism of substrate inhibition in cellulose synergistic degradation. European journal of biochemistry, 268(16):4520-6.
Vidmantiene D, Juodeikeiene G, Basinskiene L. (2006) Technical ethanol production from waste of cereals and its products using a complex enzyme preparation. J. of the Sci. of Foo and Agric. 86 : 1732-1736,
Wyman C E, Dale B E, Elander R T, Holtzapple M, Ladisch M R, Lee Y Y. (2005). Comparative sugar recovery data from laboratory scale application of leading pretreatment technologies to corn stover. Bioresource technology, 96(18):2026-32.

1. A method for treating biomass comprising the steps of:
(a) Providing at least an enzyme mixture obtained from Penicillium funiculosum deposited under Budapest treaty in the International Mycological Institute under the number IMI 378536.
(b) Providing plant biomass
(c) Contacting the enzyme mixture of step (a) and the biomass of step (b) under conditions wherein the saccharification of the biomass occurs.

2. The method for treating biomass according to any claim 1 in which the biomass is submitted to at least a pretreatment step before contacting with the enzyme mixture of step (a).

3. The method of claim 2 in which the pretreatment step is a chemical pretreatment consisting of putting the biomass into a sulfuric acid bath at 98° C., the sulfuric acid being present in a concentration of 3 g/liter.

4. The method of claim 2 in which the pretreatment step is a mechanical pretreatment consisting of crushing the biomass.

5. A method for producing bioethanol comprising the steps of:
(a) Providing at least an enzyme mixture obtained from Penicillium funiculosum deposited under Budapest treaty in the International Mycological Institute under the number IMI 378536.
(b) Providing biomass
(c) Contacting the enzyme mixture of step (a) and the biomass of step (b) under conditions wherein the saccharification of the plant waste product occurs.
(d) Fermenting the product obtained pursuant step (c).

6. The method according to claims 1 in which the enzyme mixture is provided as an isolated pure enzyme preparation.

7. The method according to claims 1 in which the enzyme mixture is provided as a crude enzyme preparation.

8. The method according to claim 1 biomass is chosen from wheat straw, wheat bran, hemp fibers or stalk of peeled hemp.

9. Use of a composition comprising an enzyme mixture obtained from Penicillium funiculosum deposited under Budapest treaty in the International Mycological Institute under the number IMI 378536 in for the saccharification of biomass.

10. A processed biomass comprising an enzyme mixture obtained from Penicillium funiculosum deposited under Budapest treaty in the International Mycological Institute under the number IMI 378536.
